(19) United States
(12) Patent Application Publication
(10) Pub. No.: US 2013/0052632 A1
(43) Pub. Date: Feb. 28, 2013

(54) METHOD FOR SENSING A CHEMICAL

(71) Applicants: Timothy Joseph Nicholas Carter, Sittingbourne (GB); Steven Andrew Ross, Sittingbourne (GB)

(72) Inventors: Timothy Joseph Nicholas Carter, Sittingbourne (GB); Steven Andrew Ross, Sittingbourne (GB)

(73) Assignee: VIVACTA LIMITED, Sittingbourne, Kent (GB)

(21) Appl. No.: 13/520,484

(22) PCT Filed: Jan. 4, 2011
(86) PCT No.: PCT/GB2011/050002

§ 371 Date: Nov. 7, 2012

Related U.S. Application Data
(60) Provisional application No. 61/292,003, filed on Jan. 4, 2010.

(30) Foreign Application Priority Data
Jan. 15, 2010 (GB) 1000643.5

(51) Int. Cl. G01N 25/20; C12M 1/42; B82Y 15/00

(52) U.S. Cl. 435/5; 436/501; 435/7.4; 435/7.1; 435/287.2; 422/430; 977/773; 977/920

(57) ABSTRACT

This invention relates to a method for detecting an analyte in a sample. The method comprises the steps of exposing the sample to a transducer having a pyroelectric or piezoelectric element and electrodes which is capable of transducing a change in energy to an electrical signal, the transducer having at least one reagent proximal thereto, the reagent having a binding site which is capable of binding the analyte or a complex or derivative of the analyte, wherein at least one of the analyte or the complex or derivative of the analyte has a label attached thereto which is capable of absorbing the electromagnetic radiation generated by the radiation source to generate energy by non-radiative decay; irradiating the reagent with a series of pulses of electromagnetic radiation, transducing the energy generated into an electrical signal, detecting the electrical signal and the time delay between each pulse of electromagnetic radiation from the radiation source and the generation of the electric signal. The time delay between each of the pulses of electromagnetic radiation and the generation of the electric signal corresponds to the position of the analyte at any of one or more positions at different distances from the surface of the transducer. The label is a nanoparticle comprising polypyrrole or a derivative thereof. The invention also provides a kit suitable for performing this method.

The present invention relates to a method for sensing a chemical, and in particular a method employing a chemical sensing device according to WO 2004/090512.

The monitoring of analytes in solution, such as biologically important compounds in bioassays, has a broad applicability. Accordingly, a wide variety of analytical and diagnostic devices are available. Many devices employ a reagent which undergoes an eye-detectable colour change in the presence of the species being detected. The reagent is often carried on a test strip and optics may be provided to assist in the measurement of the colour change.

WO 90/13017 discloses a pyroelectric or other thermoelectric transducer element in a strip form. Thin film electrodes are provided and one or more reagents are deposited on the transducer surface. The reagent undergoes a selective colorimetric change when it comes into contact with the species being detected. The device is then typically inserted into a detector where the transducer is illuminated usually through the transducer by an LED light source and light absorption by the reagent is detected as microscopic heating at the transducer surface. The electrical signal output from the transducer is processed to derive the concentration of the species being detected.

The system of WO 90/13017 provides for the analysis of species which produce a colour change in the reagent on reaction or combination with the reagent. For example, reagents include pH and heavy metal indicator dyes, reagents (e.g. o-cresol in ammoniacal copper solution) for detecting aminophenol in a paracetamol assay, and a tetrazolium dye for detecting an oxidoreductase enzyme in an enzyme-linked immuno-sorbent assay (ELISA). However, while this system is useful in certain applications, it has been considered suitable only for analysis where the species being analysed generates a colour change in the reagent since it is the reagent which is located on the surface of the transducer. Therefore, this system cannot be applied to the analysis of species which do not cause a colour change in the reagent or when the colour change is not on the surface of the transducer. In the field of bioassays, this gives the system limited applicability.

WO 2004/090512 discloses a device based on the technology disclosed in WO 90/13017, but relies on the finding that energy generated by non-radiative decay in a substance on irradiation with electromagnetic radiation may be detected by a transducer even when the substance is not in contact with the transducer, and that the time delay between the irradiation with electromagnetic radiation and the electrical signal produced by the transducer is a function of the distance of the substance from the surface of the film. This finding provided a device capable of “depth profiling” which allows the device to distinguish between an analyte bound to the surface of the transducer and an analyte in the bulk liquid. This application therefore discloses a device which is able to be used in assays, typically bioassays, without having to carry out a separate washing step between carrying out a binding event and detecting the results of that event.

However, there remains a need for systems providing improved sensitivity and selectivity. One such approach out of many has been to focus on the nature of the label. For example, WO 2007/141581 discloses an improved method and kit employing the device described in WO 2004/090512 in which the label is a nanoparticle comprising a non-conducting core material and at least one metal shell layer. The preferred label described therein is a nanoparticle comprising a gold-plated monodisperse colloidal silica. The benefit of this label is that the surface properties of the particle are similar to colloidal gold particles, but the overall density of the particle is less, thus any interference due to sedimentation effects is reduced. Otherwise, carbon particles tend to be preferred, since carbon particles suffer even less from sedimentation and are also very good absorbers of electromagnetic radiation across the visible and infra-red spectrum

Accordingly, the present invention provides a method for detecting an analyte in a sample, comprising the steps of exposing the sample to a transducer having a pyroelectric or piezoelectric element and electrodes which is capable of transducing a change in energy to an electrical signal, the transducer having at least one reagent proximal thereto, the reagent having a binding site which is capable of binding the analyte or a complex or derivative of the analyte, wherein at least one of the analyte or the complex or derivative of the analyte has a label attached thereto which is capable of absorbing the electromagnetic radiation generated by the radiation source to generate energy by non-radiative decay;

irradiating the reagent with a series of pulses of electromagnetic radiation,
transducing the energy generated into an electrical signal;
detecting the electrical signal and the time delay between each pulse of electromagnetic radiation from the radiation source and the generation of the electric signal, wherein the time delay between each of the pulses of electromagnetic radiation and the generation of the electric signal corresponds to the position of the analyte at any of one or more positions at different distances from the surface of the transducer,
wherein the label is a nanoparticle comprising polypyrrole or a derivative thereof.

The present invention also provides a kit suitable for performing the above-described method. The kit comprises

(i) a device for detecting energy generated by non-radiative decay in an analyte or a complex or derivative of the analyte on irradiation with electromagnetic radiation comprising
a radiation source adapted to generate a series of pulses of electromagnetic radiation,
a transducer having a pyroelectric or piezoelectric element and electrodes which is capable of transducing the energy generated by the substance into an electrical signal,
at least one reagent proximal to the transducer, the reagent having a binding site which is capable of binding the analyte or the complex or derivative of the analyte, and
a detector which is capable of detecting the electrical signal generated by the transducer,
wherein the detector is adapted to determine the time delay between each pulse of electromagnetic radiation from the radiation source and the generation of the electric signal; and
(ii) an analyte or a complex or a derivative of the analyte which has a label attached thereto which is capable of absorbing the electromagnetic radiation generated by the radiation source to generate energy by non-radiative decay, wherein the label is a nanoparticle comprising polypyrrole or a derivative thereof.

The particles of the present invention show a surprisingly high sensitivity and selectivity whilst being relatively straight-forward to prepare.

The present invention will now be described with reference to the drawings, in which:

FIG. 1 shows a schematic representation of the chemical sensing device of WO 2004/090512 which is used with the present invention;

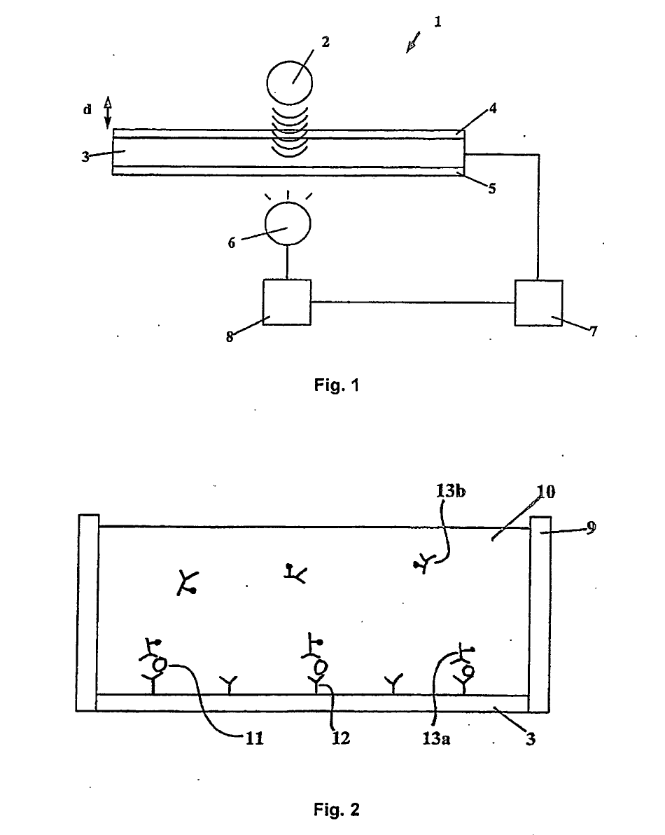

FIG. 2 shows a sandwich immunoassay using the device of the present invention;

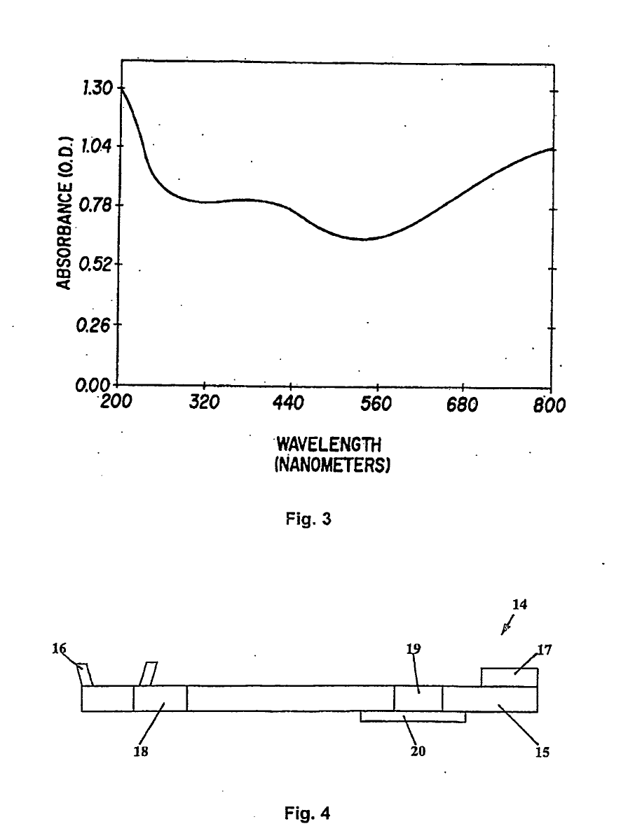

FIG. 3 shows the absorption spectrum of polypyrrole particles in accordance with the present invention at wavelengths of 200 to 800 nm;

FIG. 4 shows a lateral flow assay device in accordance with the present invention;

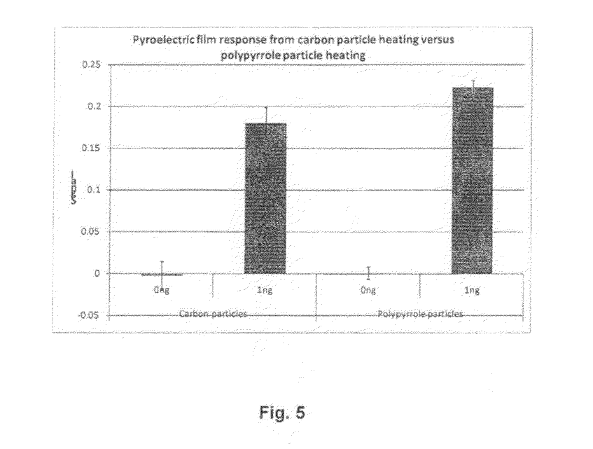

FIG. 5 shows the results of a TSH assay using carbon particles (comparative) and polypyrrole particles (present invention) as the signal-generating labels;

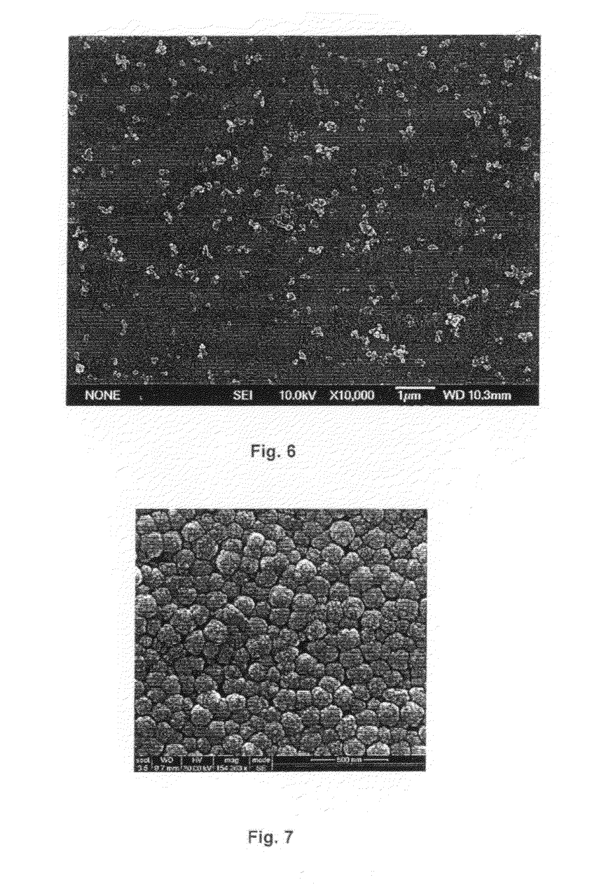

FIG. 6 shows an SEM image of antibody-coated carbon particles bound to the surface of a sensor; and

FIG. 7 shows an SEM image of polypyrrole particles in accordance with the present invention.

FIG. 1 shows a chemical sensing device 1 for use in accordance with the present invention which relies on heat generation in a substance 2 on irradiation of the substance 2 with electromagnetic radiation. FIG. 1 shows the chemical sensing device 1 in the presence of a substance 2. The device 1 comprises a pyroelectric or piezoelectric transducer 3 having electrode coatings 4,5. The transducer 3 is preferably a poled polyvinylidene fluoride film. The electrode coatings 4,5 are preferably formed from indium tin oxide having a thickness of about 35 nm, although almost any thickness is possible from a lower limit of 1 nm below which the electrical conductivity is too low and an upper limit of 100 nm above which the optical transmission is too low (it should not be less than 95% T). A substance 2 is held proximal to the piezoelectric transducer 3 using any suitable technique, shown here attached to the upper electrode coating 4. The substance may be in any suitable form and a plurality of substances may be deposited. A key feature of the present invention is that the substance 2 generates heat when irradiated by a source of electromagnetic radiation 6, such as light, preferably visible light. The light source may be, for example, an LED. The light source 6 illuminates the substance 2 with light of the appropriate wavelength (e.g. a complementary colour). Although not wishing to be bound by theory, it is believed that the substance 2 absorbs the light to generate an excited state which then undergoes non-radiative decay thereby generating energy, indicated by the curved lines in FIG. 1. This energy is primarily in the form of heat (i.e. thermal motion in the environment) although other forms of energy, e.g. a shock wave, may also be generated. The energy is, however, detected by the transducer and converted into an electrical signal. The device of the present invention is calibrated for the particular substance being measured and hence the precise form of the energy generated by the non-radiative decay does not need to be determined. Unless otherwise specified the term “heat” is used herein to mean the energy generated by non-radiative decay. The light source 6 is positioned so as to illuminate the substance 2. Preferably, the light source 6 is positioned opposite the transducer 3 and electrodes 4,5 and the substance 2 is illuminated through the transducer 3 and electrodes 4,5. The light source may be an internal light source within the transducer in which the light source is a guided wave system. The wave guide may be the transducer itself or the wave guide may be an additional layer attached to the transducer.

The energy generated by the substance 2 is detected by the transducer 3 and converted into an electrical signal. The electrical signal is detected by a detector 7. The light source 6 and the detector 7 are both under the control of the controller 8. The light source 6 generates a series of pulses of light (the term “light” used herein means any form of electromagnetic radiation unless a specific wavelength is mentioned) which is termed “chopped light”. In principle, a single flash of light, i.e. one pulse of electromagnetic radiation, would suffice to generate a signal from the transducer 3. However, in order to obtain a reproducible signal, a plurality of flashes of light are used which in practice requires chopped light. The frequency at which the pulses of electromagnetic radiation are applied may be varied. At the lower limit, the time delay between the pulses must be sufficient for the time delay between each pulse and the generation of an electrical signal to be determined. At the upper limit, the time delay between each pulse must not be so large that the period taken to record the data becomes unreasonably extended. Preferably, the frequency of the pulses is from 1-50 Hz, more preferably 1-10 Hz and most preferably 2 Hz. This corresponds to a time delay between pulses of 20-1,000 ms, 100-1,000 ms and 500 ms, respectively. In addition, the so-called “mark-space” ratio, i.e. the ratio of on signal to off signal is preferably one although other ratios may be used without deleterious effect. There are some benefits to using a shorter on pulse with a longer off signal, in order to allow the system to approach thermal equilibrium before the next pulse perturbs the system. In one embodiment, a light pulse of 1-50 ms, preferably 10 ms, followed by a relaxation time of 300-700 ms, preferably 490 ms allows a more precise measurement of particles bound directly to the surface. Sources of electromagnetic radiation which produce chopped light with different frequencies of chopping or different mark-space ratios are known in the art. The detector 7 determines the time delay (or “correlation delay”) between each pulse of light from light source 6 and the corresponding electrical signal detected by detector 7 from transducer 3. The applicant has found that this time delay is a function of the distance, d.

Any method for determining the time delay between each pulse of light and the corresponding electrical signal which provides reproducible results may be used. Preferably, the time delay is measured from the start of each pulse of light to the point at which a maximum in the electrical signal corresponding to the absorption of heat is detected as by detector 7.

The finding that the substance 2 may be separated from the transducer surface and that a signal may still be detected is surprising since the skilled person would have expected the heat to be dispersed into the surrounding medium and hence be undetectable by the transducer 3 or at least for no meaningful signal to be received by the transducer. The applicant has found, surprisingly, that not only is the signal detectable through an intervening medium capable of transmitting energy to the transducer 3, but that different distances, d, may be distinguished (this has been termed “depth profiling”) and that the intensity of the signal received is proportional to the concentration of the substance 2 at the particular distance, d, from the surface of the transducer 3. Moreover, the applicant has found that the nature of the medium itself influences the time delay and the magnitude of the signal at a given time delay. These findings provide a wide number of new applications for chemical sensing devices employing a transducer.

In one embodiment, the present invention employs a device as defined above wherein the substance is an analyte or a complex or derivative of the analyte, the device being used for detecting the analyte in a sample, the device further comprising at least one reagent proximal to the transducer, the reagent having a binding site which is capable of binding the analyte or the complex or derivative of the analyte, wherein the analyte or the complex or derivative of the analyte is capable of absorbing the electromagnetic radiation generated by the radiation source to generate heat, wherein, in use, the heat generated is transduced into an electrical signal by the transducer and is detected by the detector, and the time delay between each of the pulses of electromagnetic radiation and the generation of the electric signal corresponds to the position of the analyte at any of one or more positions at different distances from the surface of the transducer. The present invention provides a method using the device. Such a method has applicability in, for example, immunoassays and nucleic acid-based assays. In a preferred example of an immunoassay, the reagent is an antibody and the analyte may be considered to be an antigen.

In a typical immunoassay, an antibody specific for an antigen of interest is attached to a polymeric support such as a sheet of polyvinylchloride or polystyrene. A drop of cell extract or a sample of serum or urine is laid on the sheet, which is washed after formation of the antibody-antigen complex. Antibody specific for a different site on the antigen is then added, and the sheet is again washed. This second antibody carries a label so that it can be detected with high sensitivity. The amount of second antibody bound to the sheet is proportional to the quantity of antigen in the sample. This assay and other variations on this type of assay are well known, see, for example, “The Immunoassay Handbook, 2nd Ed.” David Wild, Ed., Nature Publishing Group, 2001. The device of the present invention may be used in any of these assays. Competitive, displacement and anti-complex antibody immunoassays also warrant particular mention.

By way of example, FIG. 2 shows a typical capture antibody assay using the device of the present invention. A device includes a transducer 3 and a well 9 for holding a liquid 10 containing an analyte 11 dissolved or suspended therein. The transducer 3 has a number of reagents, i.e. antibody 12, attached thereto. The antibody 12 is shown attached to the film in FIG. 2 and this attachment may be via a covalent bond or by non-covalent adsorption onto the surface, such as by hydrogen bonding. Although the antibody is shown as attached to the transducer, any technique for holding the antibody 12 proximal to the transducer 3 is applicable. For example, an additional layer may separate the antibody 12 and the transducer 3, such as a silicone polymer layer, or the antibody could be attached to inert particles and the inert particles are then attached to the transducer 3. Alternatively, the antibody 12 could be entrapped within a gel layer which is coated onto the surface of the transducer 3.

In use, the well is filled with liquid 10 (or any fluid) containing an antigen 11. The antigen 11 then binds to antibody 12. Additional labelled antibody 13 is added to the liquid and a so-called “sandwich” complex is formed between the bound antibody 12, the antigen 11 and the labelled antibody 13. By “labelled” antibody we mean an antibody which is conjugated to a secondary species, for example a light-absorbing particle such as those described above (carbon, polypyrrole etc). An excess of labelled antibody 13 is added so that all of the bound antigen 11 forms a sandwich complex. The sample therefore contains bound labelled antibody 13a and unbound labelled antibody 13b free in solution.

During or following formation of the sandwich complex, the sample is irradiated using a series of pulses of electromagnetic radiation, such as light. The time delay between each pulse and the generation of an electrical signal by the transducer 3 is detected by a detector. The appropriate time delay is selected to measure only the heat generated by the bound labelled antigen 13a. Since the time delay is a function of the distance of the label from the transducer 3, the bound labelled antibody 13a may be distinguished from the unbound labelled antigen 13b. This provides a significant advantage over the conventional sandwich immunoassay in that it removes the need for washing steps. In a conventional sandwich immunoassay, the unbound labelled antibody must be separated from the bound labelled antibody before any measurement is taken since the unbound labelled antigen interferes with the signal generated by the bound labelled antigen. However, on account of the “depth profiling” provided by the present invention, bound and unbound labelled antigen may be distinguished. Indeed, the ability to distinguish between substances proximal to the transducer and substances in the bulk solution is a particular advantage of the present invention.

It has been found that particularly advantageous results may be obtained when the labelled reagent is a particle comprising polypyrrole or derivatives thereof.

Polypyrrole may be prepared by the chemical oxidation of pyrrole, usually by iron(III) chloride under aqueous acidic conditions. In order to generate stable particles, the polymerisation is commonly carried out in the presence of a stabilising agent, normally a water-soluble polymer such as polyvinyl alcohol or polyvinyl pyrrolidinone. Derivatives of polypyrrole are also contemplated by the present invention. Suitable derivatives include polypyrrole in which one or more of the pyrrole rings is substituted with one or more substituents selected from C₁₋₆-alkyl, aryl (e.g. phenyl), aryl-C₁₋₆-alkyl (e.g. benzyl) and combinations thereof. The pyrrole is preferably substituted via the N-position of the pyrrole ring. The weight average molecular weight of the polypyrrole or derivative thereof is preferably in the region of 10 million to 20 billion grams per mol, corresponding to a particle diameter of between approximately 20 nm and 500 nm.

The particles are preferably prepared by emulsion polymerisation. It is believed that surface tension effects result in the formation of small droplets in the emulsion which encourages the formation of uniform, spherical particles. It should be noted that carbon particles cannot be prepared in this manner.

The particles may be composed solely of polypyrrole or derivatives thereof, or may include a combination of polypyrrole or derivatives thereof with another material. For example, the particles may be composite particles where pyrrole monomers or derivatives thereof are polymerised in the presence of other particles, such as silica particles to generate polypyrrole-silica composites, which form stable colloids in the absence of stabilising agents.

The polymer particles may also be subjected to chemical modification, for example to help stability and/or to aid antibody conjugation. This chemical modification may take place either prior to, during, or after the particle formation. Chemical modification prior to polymerisation may be achieved by the addition of side-chains to the monomer precursors. Modification during the polymerisation can be via co-polymerisation of two different monomer units, either of which may also contain side-chains. In this embodiment, the nanoparticle of the present invention comprises a co-polymer of pyrrole or a derivative thereof and a co-monomer. Modification after polymerisation can be carried out by a different number of methods. For example, polypyrrole particles can react by nucleophilic attack of their surface amine groups on electrophiles, such as bromoacetyl bromide. There are numerous methods for subsequent covalent conjugation of antibodies to such materials, either through amino acid side chains or via the carbohydrate group of the Fc domain of the antibody. One particularly attractive route is by functionalisation of the polypyrrole particle with maleimide groups, followed by covalent attachment of antibody fragments which contain free thiol groups. Methods are known for the generation of such antibody fragments, known as F(ab) fragments by enzymatic digestion of the antibody, followed by selective reduction of the hinge disulfide bond.

Suitable labels are described in U.S. Pat. No. 5,252,459, U.S. Pat. No. 5,681,754 and M. R. Pope et al. Bioconjugate Chemistry 1996, 7, 436.

Preferably, the present invention uses a nanoparticle having a particle size of 20-500 nm, more preferably 100-200 nm. Outside this range, smaller particles do not generate enough heat, whereas larger particles diffuse too slowly to the surface of the transducer. By particle size is meant the diameter of the particle at its widest point. Preferably the particle is substantially spherical. Particles having a diameter 100 nm have a weight average molecular weight of approximately 300-400 million grams per mol.

The labelled analyte, complex or derivative, and any one or more additional reagents are preferably stored in a chamber incorporated into the device employed in the present invention.

The analyte is typically a protein, such as a protein-based hormone, although smaller molecules, such as drugs, may be detected. The analyte may also be part of a larger particle, such as a virus, a bacterium, a cell (e.g. a red blood cell) or a prion.

As a further example of known immunoassays, the present invention may be applied to competitive assays in which the electrical signal detected by the detector is inversely proportional to the presence of an unlabelled antigen in the sample. In this case, it is the amount of the unlabelled antigen in the sample which is of interest.

In a competitive immunoassay, an antibody is attached to the transducer as shown in FIG. 2. A sample containing the antigen is then added. However, rather than adding a labelled antibody, a known amount of labelled antigen is added to the solution. The labelled and unlabelled antigens then compete for binding to the antibodies attached to the transducer 3. The concentration of the bound labelled antigen is then inversely proportional to the concentration of bound unlabelled antigen and hence, since the amount of labelled antigen is known, the amount of unlabelled antigen in the initial solution may be calculated. The same labels specified with reference to the antibodies may also be used with the antigens.

In an embodiment of the present invention, the analyte being detected may be present in a sample of whole blood. In many conventional assays, the presence of other components of the blood in solution or suspension, such as red blood cells, interferes with the detection of the particular analyte of interest. However, in the device of the present invention, since only the signal at a known distance from the transducer 3 is determined, the other components of the blood which are free in solution or suspension do not interfere with the detection. This simplifies the analysis of a blood sample since a separate separation step is not required. An apparatus for measuring analyte levels in a blood sample preferably comprises a hand-held portable reader and a disposable device containing the piezoelectric film. A small sample of blood (about 10 μL) is obtained and transferred to a chamber within the disposable device. One side of the chamber is made from the piezoelectric film coated with an antibody capable of binding to the analyte of interest. An additional solution may then be added containing, for example, labelled antibody or a known concentration of labelled antigen as described above. The reaction is allowed to proceed and the disposable device is then inserted into the reader which activates the measurement process. The results of the assay are then indicated on a display on the reader. The disposable device containing the piezoelectric film is then removed and discarded.

Advantageously, the nanoparticles of the present invention absorb radiation quite uniformly across the visible spectrum and into the infra-red region, and thus absorb well in the “blood window” of around 600-900 nm. The absorption spectrum of polypyrrole particles at 0.00216% solids over wavelengths of 200 to 800 nm is shown in FIG. 3.

A potential source of background interference is the settling of suspended particles on to the surface of the pyroelectric or piezoelectric transducer, including antibody label particles and cellular components of the sample. This source of interference may be avoided by positioning the transducer above the bulk solution, e.g. on the upper surface of the reaction chamber. Thus, if any settling occurs, it will not interfere with the transducer. Alternatively, the particles could be less dense than the medium and hence float to the surface of the bulk solution rather than settling on the surface of the transducer. This and other modifications are included in the scope of the present invention.

In another embodiment, the device of the present invention is applied to lateral-flow analysis. This has particular application for the detection of human chorionic gonadotrophin (HCG) in pregnancy testing.

FIG. 4 shows a simplified lateral flow device 14 in accordance with the present invention. The device has a filter paper or other absorber 15 containing a sample receiver 16 and a wick 17 together with first and second zones 18 and 19 containing unbound and bound antibodies (i.e. unbound and bound to the filter paper or other absorber 15), respectively, capable of binding to HCG. The device also contains a piezoelectric film 20 proximal to the second zone 19. A sample of urine or serum is added to the sample receiver 16 which then travels along the absorber 15 to the wick 17. The first zone 18 contains a labelled antibody to HCG and as the sample passes through the first zone 18, if HCG is present in the sample, the labelled antibody to HCG is picked up by the sample. As the sample passes from the first zone 18 to the second zone 19, the antigen and antibody form a complex. At the second zone 19, a second antibody is attached either to the absorber 15 or the piezoelectric film 20 which is capable of binding the antigen-antibody complex. In a conventional lateral-flow analysis such as a pregnancy tester, a positive result produces a colour change at the second zone 19. However, the conventional lateral-flow analysis is restricted to clear samples and is essentially suitable only for a positive or negative i.e. yes/no, result. The device of the present invention, however, uses a piezoelectric film 20. Since only the sample at the predetermined distance from the film is measured, contaminants in the bulk sample will not affect the reading. Moreover, the sensitivity of the piezoelectric film provides a quantification of the result. Quantification of the result provides a broader applicability to the lateral-flow analysis and also distinguishing between different quantities of antigens reduces the number of erroneous results.

The device of the present invention is not restricted to detecting only one analyte in solution. Since the device provides “depth profiling” different analytes may be detected by employing reagents which selectively bind each analyte being detected wherein the reagents are different distances from the surface of the transducer 3. For example, two analytes may be detected using two reagents, the first reagent being positioned at a first distance from the film and the second reagent being positioned at a second distance from the film. The time delay between each pulse of electromagnetic radiation and the generation of electrical signal will be different for the two analytes bound to the first and second reagents.

As well as providing different depths, multiple tests may be carried out using different types of reagents, e.g. different antibodies, at different parts of the transducer. Alternatively, or in addition, multiple tests may be carried out using reagents/analytes which respond to different wavelengths of electromagnetic radiation. Multiple tests can also be carried out using only one electrical connection to the transducer, by illuminating different parts of the transducer sequentially and interrogating the outputs sequentially.

The substance generating the heat may be on the surface of the film, however, preferably the substance is at least 5 nm from the surface of the film and, preferably, the substance is no more than 500 μm from the surface of the film. By selecting a suitable time delay, however, a substance in the bulk solution may also be measured.

As alternatives to antibody-antigen reactions, the reagent and analyte may be a first and second nucleic acid where the first and second nucleic acids are complementary, or a reagent containing avidin or derivatives thereof and an analyte containing biotin or derivatives thereof, or vice versa. The system is also not limited to biological assays and may be applied, for example, to the detection of heavy metals in water. The system also need not be limited to liquids and any fluid system may be used, e.g. the detection of enzymes, cells and viruses etc. in the air.

As described hereinabove, the applicant has found that the time delay between each pulse of electromagnetic radiation in the generation of an electric signal in the transducer is proportional to the distance of the substance from the film. Moreover, the applicant has found that the time delay depends on the nature of the medium itself. Initially, it was surprising that a liquid medium does not totally dampen the signal. However, the applicant has found that changes in the nature of the medium can alter the time delay (i.e. until signal maximum is reached), the magnitude of the signal and the waveform of the signal, (i.e. the variation of response over time).

These changes in the nature of the medium may be due to, amongst other things variations in the thickness of the medium, the elasticity of the medium, the hardness of the medium, the density of the medium, the deformability of the medium, the heat capacity of the medium or the speed at which sound/shock waves may be propagated through the medium.

The present invention will now be described with reference to the following examples which are not intended to be limiting.

### EXAMPLES

A poled polyvinylidene fluoride bimorph, coated in indium tin oxide, was used as the sensing device in the following examples.

### Example 1

### Optical Properties of Polypyrrole Particles

Suspensions of carbon particles (of nominal size 200 nm) and suspensions of polyvinylpyrrolidinone-stabilised polypyrrole particles (of nominal size 200 nm) were both prepared at a concentration of 0.001% solids. The optical density of these solutions was then measured at a wavelength of 690 nm and a path-length of 0.005 m. Absorbance values of 0.190 and 0.165 were obtained for the carbon and polypyrrole suspensions respectively.

### Example 2

### Reference Example

### Preparation of Anti-TSH Coated Carbon Particles

Carbon colloid (at a concentration of 0.1% solids w/v) was prepared by sonication of SB4 carbon particles from Evonik Degussa GmbH, Dusseldorf. To this solution was then added a monoclonal anti-TSH antibody at a concentration of 100 μg/mL and the solution was incubated at 20° C. for 24 hours. The carbon colloid was then washed 3 times by centrifugation at 13,000 g for 45 mins, followed by removal of the supernatant and resuspension in 10 mM phosphate buffer pH 7.2.

### Example 3

### Preparation of Anti-TSH Coated Polypyrrole Particles

Polypyrrole particles of mean diameter 195 nm were prepared by the oxidation of pyrrole by FeCl₃ in the presence of polyvinylpyrrolidinone. The resultant particles were diluted to a concentration of 0.1% solids w/v in phosphate buffer pH 7.2 containing 0.05% Tween® 20. To this solution was then added a monoclonal anti-TSH antibody at a concentration of 100 μg/mL and the solution was incubated at 20° C. for 24 hours. The polypyrrole particles were then washed 3 times by centrifugation at 13,000 g for 45 mins, followed by removal of the supernatant and resuspension in 10 mM phosphate buffer pH 7.2.

### Example 4

### TSH Assay

TSH assays were carried out using the system described hereinabove with reference to WO 2004/090512 and WO 2007/141581

30 μL of human plasma was mixed with the anti-TSH coated particles (either carbon or polypyrrole particles) to give a final particle concentration of 0.005% solids w/v. Samples contained differing levels of TSH hormone, including samples which nominally contained no TSH, were used. After mixing, the sample was then pumped into a series of three chamber's, the top and bottom of which were formed from a piece of piezoelectric PVDF polymer and an injection-moulded acrylic base, respectively. The top and the base are separated by a spacer-tape constructed of 150 micron thick polyester, coated with 25 microns of adhesive on each side. Thus the total depth of the chamber is approximately 200 microns. The chambers are substantially circular, with a diameter of approximately 6 mm. The underside of the piezofilm, adjacent to the sample, is conformally coated with parylene C of approximate thickness 1 micron using methods known in the art (see WO 2009/141637). Antibodies are also coated on top of the parylene C surface using methods known in the art. The piezofilm surface in each of the three chambers is coated with a different antibody. The first chamber (designated the “negative control” chamber) is coated in an antibody that should have no affinity for TSH or for any other components in the system. The second chamber (designated the “measurement” chamber) is coated with a matched monoclonal antibody to TSH (i.e. an antibody which recognises a different epitope on the TSH molecule from the antibody which has been used to coat the carbon/polypyrrole particles). The third chamber (designated the “positive control” chamber) is coated with a polyclonal goat anti-mouse antibody, which binds the particles in the absence (or presence) of TSH, and hence gives an indication that the system is working. During the measurement process, light of wavelength 690 nm, from an LED source is pulsed into the system and the piezoelectric response from the PVDF film is amplified and measured. Binding of carbon/polypyrrole particles to the surface of the piezofilm in the “measurement” chamber during the measurement leads to increased heating of the PVDF upon illumination, generating a signal which can be quantified. The signal is interrogated in order to obtain the greatest discrimination of heating due to bound particles at the surface of the transducer relative to unbound particles in the bulk solution. The greater the signal, the more TSH in the sample. The negative and positive control chambers can also be used as internal references (standards, or controls), although they do not have to be used in this fashion. The signal is measured as the rate of change of electrical charge output over a period of 10 minutes, with the charge being converted to an arbitrary digital response.

A number of repeat measurements (10 of each) were carried out on human plasma samples containing TSH at concentrations of 1 ng/mL or 0 ng/mL (nominal), using both carbon particles and polypyrrole particles as the signal-generating label. The results are shown in FIG. 5, with 1 standard deviation error bars included. It can be seen clearly that there is an enhanced signal from the polypyrrole particle system in comparison to the carbon particle system. Additionally, the precision in the polypyrrole particle system is better than the carbon particle system.

Since the carbon particles have a higher extinction coefficient compared to the polypyrrole particles and hence absorb more light (see Example 1), it would be expected that the carbon particles should give a higher signal in the presence of TSH. However, the polypyrrole particles work better in the present system, which monitors the kinetic binding of colloidal particles to a solid surface.

Without wishing to be bound by theory, it is speculated that the polypyrrole particles may provide an improved performance over carbon particles despite their lower light absorption properties because polypyrrole particles tend to have a quite uniform shape, whereas carbon particles tend to have quite uneven shapes as shown in FIG. 6, which shows an SEM image of antibody-coated carbon particles bound to the surface of the sensor in the current device. In contrast the SEM image in FIG. 7 shows the more uniform shape and size distribution of polypyrrole particles (in this image the particles are not bound to the surface of the film). The shape of the particles may reduce steric hindrance as they approach the surface, thus increasing the rate of binding of particle to surface.

It is further speculated that a complication of the presently described assay system is that there may be some interference from the general heating of unbound particles in the bulk solution when trying to measure those particles which are specifically bound at the surface. This interference is magnified if there is unwanted sedimentation of the particles in the bulk solution, or the sedimentation of the particles in the bulk solution is less predictable. Thus, the improved shape and monodispersity of the polypyrrole particles compared to carbon particles may help to reduce this interfering signal, and hence improve the precision of the measurement.

1. A method for detecting an analyte in a sample, comprising the steps of
exposing the sample to a transducer having a pyroelectric or piezoelectric element and electrodes which is capable of transducing a change in energy to an electrical signal, the transducer having at least one reagent proximal thereto, the reagent having a binding site which is capable of binding the analyte or a complex or derivative of the analyte, wherein at least one of the analyte or the complex or derivative of the analyte has a label attached thereto which is capable of absorbing the electromagnetic radiation generated by the radiation source to generate energy by non-radiative decay;
irradiating the reagent with a series of pulses of electromagnetic radiation,
transducing the energy generated into an electrical signal;
detecting the electrical signal and the time delay between each pulse of electromagnetic radiation from the radiation source and the generation of the electric signal, wherein the time delay between each of the pulses of electromagnetic radiation and the generation of the electric signal corresponds to the position of the analyte at any of one or more positions at different distances from the surface of the transducer,
wherein the label is a nanoparticle comprising polypyrrole or a derivative thereof.

2. A method as claimed in claim 1, wherein the label is a nanoparticle composed of a co-polymer of pyrrole or a derivative thereof and a co-monomer.

3. A method as claimed in claim 2, wherein the label is a composite nanoparticle of polypyrrole or a derivative thereof, or the co-polymer, and another material.

4. A method as claimed in claim 3, wherein the label is a polypyrrole-silica composite.

5. A method as claimed in claim 1, wherein the reagent is an antibody.

6. A method as claimed in claim 1, wherein the method is carried out without removing the sample from the transducer between the steps of exposing the sample to the transducer and irradiating the reagent.

7. A kit comprising
(i) a device for detecting energy generated by non-radiative decay in an analyte or a complex or derivative of the analyte on irradiation with electromagnetic radiation comprising
a radiation source adapted to generate a series of pulses of electromagnetic radiation,
a transducer having a pyroelectric or piezoelectric element and electrodes which is capable of transducing the energy generated by the substance into an electrical signal,
at least one reagent proximal to the transducer, the reagent having a binding site which is capable of binding the analyte or the complex or derivative of the analyte, and
a detector which is capable of detecting the electrical signal generated by the transducer,
wherein the detector is adapted to determine the time delay between each pulse of electromagnetic radiation from the radiation source and the generation of the electric signal; and
(ii) an analyte or a complex or a derivative of the analyte which has a label attached thereto which is capable of absorbing the electromagnetic radiation generated by the radiation source to generate energy by non-radiative decay, wherein the label is a nanoparticle comprising polypyrrole or a derivative thereof.

8. A kit as claimed in claim 7, wherein the label is a nanoparticle composed of a co-polymer of pyrrole or a derivative thereof and a co-monomer.

9. A kit as claimed in claim 8, wherein the label is a composite nanoparticle of polypyrrole or a derivative thereof, or the co-polymer, and another material.

10. A kit as claimed in claim 9, wherein the label is a polypyrrole-silica composite.

11. A kit as claimed in claim 7, wherein the reagent is an antibody.

12. A kit as claimed in claim 7, wherein the reagent is adsorbed on to the transducer.

13. A kit as claimed in claim 7, wherein the device further comprises a well for holding a liquid sample containing the analyte or the complex or derivative of the analyte in contact with the transducer.
